SEQUENCE LISTING (39 sequences)

SEQ ID NO: 1 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 2 - protein, 11 aa, synthetic construct
RASQDIRSDFG

SEQ ID NO: 3 - protein, 8 aa, synthetic construct
YAASSLQS

SEQ ID NO: 4 - protein, 9 aa, synthetic construct
LQDYNYPWT

SEQ ID NO: 5 - protein, 107 aa, synthetic construct
AIQLTQSPSSLSASVGDRVTITCRASQDIRSDFGWYQQKPGKAPKLLIYAASSLQSGVPSRFSGSGSGTDFTLTISSLQPEDFATYYCLQDYNYPWTFGQGTKVEIK

SEQ ID NO: 6 - protein, 214 aa, synthetic construct
AIQLTQSPSSLSASVGDRVTITCRASQDIRSDFGWYQQKPGKAPKLLIYAASSLQSGVPSRFSGSGSGTDFTLTISSLQPEDFATYYCLQDYNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 13 aa, synthetic construct
KASGYTFTGYYLH

SEQ ID NO: 8 - protein, 17 aa, synthetic construct
WINPNSGGTNYAQKFQG

SEQ ID NO: 9 - protein, 15 aa, synthetic construct
ARDIVVLPPAIGFDY

SEQ ID NO: 10 - protein, 15 aa, synthetic construct
ARDIVVLPPAIGFDL

SEQ ID NO: 11 - protein, 122 aa, synthetic construct
QVQLVQSGAEVKKPGASVKVSCKASGYTFTGYYLHWVRQAPGQGLEWMGWINPNSGGTNYAQKFQGRVTMTRDTSISTAYMELSRLRSDDTAVYYCARDIVVLPPAIGFDYWGQGTLVTVSS

SEQ ID NO: 12 - protein, 448 aa, synthetic construct
QVQLVQSGAEVKKPGASVKVSCKASGYTFTGYYLHWVRQAPGQGLEWMGWINPNSGGTNYAQKFQGRVTMTRDTSISTAYMELSRLRSDDTAVYYCARDIVVLPPAIGFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 13 - protein, 122 aa, synthetic construct
QVQLVQSGAEVKKPGASVKVSCKASGYTFTGYYLHWVRQAPGQGLEWMGWINPNSGGTNYAQKFQGRVTMTRDTSISTAYMELSRLRSDDTAVYYCARDIVVLPPAIGFDLWGQGTLVTVSS

SEQ ID NO: 14 - protein, 448 aa, synthetic construct
QVQLVQSGAEVKKPGASVKVSCKASGYTFTGYYLHWVRQAPGQGLEWMGWINPNSGGTNYAQKFQGRVTMTRDTSISTAYMELSRLRSDDTAVYYCARDIVVLPPAIGFDLWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 15 - protein, 188 aa, synthetic construct
MKLQCVSLWLLGTILMLCSVDNHGLRRCLISTDMDHIEDSFQEIKRAIQAKDTFPNVTILSTLETLQIIKPLDVCCVTKNLLAFYVDRVFKDHQEPNPKILRKISSIANSFLYMQKTLRQCQEQRQCHCRQEATNVTRVIHDNYDQLEVRSAAVKSLGELDIFLAWIAKNHEVTSSAAAHHHHHHSGS

SEQ ID NO: 16 - protein, 13 aa, synthetic construct
QASESVYNKNWLS

SEQ ID NO: 17 - protein, 7 aa, synthetic construct
DSSDLAS

SEQ ID NO: 18 - protein, 9 aa, synthetic construct
GGSYTDTYV

SEQ ID NO: 19 - protein, 109 aa, synthetic construct
AQVLTQTPSSVSEPVGGTVTINCQASESVYNKNWLSWFQQKPGQPPKLLIYDSSDLASGVPSRFKGSGSGTHFTLTISDVQCGDAATYYCGGSYTDTYVFGGGTEVVVK

SEQ ID NO: 20 - protein, 213 aa, synthetic construct
AQVLTQTPSSVSEPVGGTVTINCQASESVYNKNWLSWFQQKPGQPPKLLIYDSSDLASGVPSRFKGSGSGTHFTLTISDVQCGDAATYYCGGSYTDTYVFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 21 - protein, 11 aa, synthetic construct
GFDLSSTYYMS

SEQ ID NO: 22 - protein, 19 aa, synthetic construct
SIVTSGRSGASYYANWAKG

SEQ ID NO: 23 - protein, 11 aa, synthetic construct
DLPAPTDDRGL

SEQ ID NO: 24 - protein, 121 aa, synthetic construct
QQLEESGGGLAKPEGSLTLTCKTSGFDLSSTYYMSWVRQAPGKGLEWIASIVTSGRSGASYYANWAKGRFTISKTSSTTVTLQMTSLAAADTASYFCVRDLPAPTDDRGLWGPGTLVVVSS

SEQ ID NO: 25 - protein, 444 aa, synthetic construct
QQLEESGGGLAKPEGSLTLTCKTSGFDLSSTYYMSWVRQAPGKGLEWIASIVTSGRSGASYYANWAKGRFTISKTSSTTVTLQMTSLAAADTASYFCVRDLPAPTDDRGLWGPGTLVVVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 26 - DNA, 639 bp, synthetic construct
  source 1..639: mol_type = other DNA
gcccaagtgctgacccagactccatcctccgtgtctgaacctgtgggaggcacagtcaccatcaattgccaggccagtgagagtgtttataataagaactggttatcctggtttcagcagaaaccagggcagcctcccaagctcctgatctatgattcatccgatctggcatctggggtcccatcgcggttcaaaggcagtggatctgggacacacttcactctcaccatcagcgacgtgcagtgtggcgatgctgccacttattactgtggaggcagttatactgatacctatgttttcggcggagggaccgaagtggtggtcaaaggggatccagtggcccccaccgtgctgattttcccaccagccgccgatcaggtcgccaccggcaccgtgacaatcgtgtgcgtggccaacaagtacttccccgacgtgaccgtgacctgggaggtggacggcaccacccagaccaccggcatcgagaacagcaagaccccccagaattctgccgactgcacctacaacctgagcagcaccctgaccctgaccagcacccagtacaacagccacaaagagtacacctgtaaagtcacccagggcaccaccagcgtggtgcagagcttcaaccggggcgactgc

SEQ ID NO: 27 - DNA, 1332 bp, synthetic construct
  source 1..1332: mol_type = other DNA
cagcagctggaggagtccgggggaggcctggccaagcctgagggatccctgacactcacctgcaaaacctctggattcgacctcagtagcacctactacatgtcctgggtccgccaggctccagggaaggggttggagtggatcgcatccattgttactagtggtcgtagtggtgcgtcttactacgcgaactgggcaaaaggccgattcaccatctccaaaacctcgtcgaccacggtgactctgcaaatgaccagtctggcagccgcggacacggcctcctatttctgtgtgagagatttaccagctcctactgatgatcgtggcttgtggggcccaggcaccctggtcgtcgtctcctcaggacagcctaaggccccctccgtgttcccgctagccccttgctgtggcgacaccccttctagcaccgtgacactgggctgcctggtgaagggctacctgcccgagcctgtgaccgtgacctggaacagcggcaccctgaccaacggcgtgagaaccttccctagcgtgcggcagagcagcggcctgtacagcctgagcagcgtggtgtccgtgaccagcagcagccagcccgtgacctgcaatgtggcccaccccgccaccaacaccaaggtggacaagaccgtggcccccagcacctgtagcaagcctacctgcccccctcctgaactgctgggcggacccagcgtgttcatcttcccacccaagcccaaggacaccctgatgatcagccggacccccgaagtgacctgcgtggtggtggacgtgtcccaggacgaccccgaggtgcagttcacctggtacatcaacaacgaacaagtgcggaccgccagacctcctctgcgggagcagcagttcaacagcaccatccgggtggtgtccaccctgcctatcgcccaccaggactggctgcggggcaaagaatttaagtgcaaggtccacaacaaggccctgcctgcccccatcgagaaaaccatcagcaaggccagaggccagcccctggaacctaaggtgtacaccatgggccctcccagagaggaactgagcagcagaagcgtgtccctgacctgcatgatcaacggcttctaccccagcgacatcagcgtggagtgggagaagaacggcaaggccgaggacaactacaagaccacccctgccgtgctggatagcgacggcagctacttcctgtacagcaagctgagcgtgcccacctctgagtggcagcggggcgacgtgttcacctgtagcgtgatgcacgaggccctgcacaaccactacacccagaagtccatcagcagatcccccggcaag

SEQ ID NO: 28 - protein, 11 aa, synthetic construct
SASASIYEYLS

SEQ ID NO: 29 - protein, 7 aa, synthetic construct
GASVLTD

SEQ ID NO: 30 - protein, 14 aa, synthetic construct
QSYNDGSSSGDAHV

SEQ ID NO: 31 - protein, 112 aa, synthetic construct
DIVMTQTPASVEAAVGGTVTIKCSASASIYEYLSWYQQKPGQRPKLLIYGASVLTDGVSSRFKGSGSGTEFTLTISDLEAADAATYYCQSYNDGSSSGDAHVFGGGTEVVVK

SEQ ID NO: 32 - protein, 216 aa, synthetic construct
DIVMTQTPASVEAAVGGTVTIKCSASASIYEYLSWYQQKPGQRPKLLIYGASVLTDGVSSRFKGSGSGTEFTLTISDLEAADAATYYCQSYNDGSSSGDAHVFGGGTEVVVKGDPVAPTVLIFPPAADQVATGTVTIVCVANKYFPDVTVTWEVDGTTQTTGIENSKTPQNSADNTYNLSSTLTLTSTQYNSHKEYTCKVTQGTTSVVQSFNRGDC

SEQ ID NO: 33 - protein, 10 aa, synthetic construct
GFPLSSYGFS

SEQ ID NO: 34 - protein, 17 aa, synthetic construct
YLDPVFGSTLSAHTVNG

SEQ ID NO: 35 - protein, 13 aa, synthetic construct
GIGYVYYGYTYDL

SEQ ID NO: 36 - protein, 122 aa, synthetic construct
QEQLKESGGGLVQPGGSLKLSCKASGFPLSSYGFSWVRQAPGKGLEWIGYLDPVFGSTLSAHTVNGRLTISSDNAQNTLYLQLNSLTAADTATYFCARGIGYVYYGYTYDLWGPGTLVTVSS

SEQ ID NO: 37 - protein, 445 aa, synthetic construct
QEQLKESGGGLVQPGGSLKLSCKASGFPLSSYGFSWVRQAPGKGLEWIGYLDPVFGSTLSAHTVNGRLTISSDNAQNTLYLQLNSLTAADTATYFCARGIGYVYYGYTYDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 38 - DNA, 648 bp, synthetic construct
  source 1..648: mol_type = other DNA
gacattgtgatgacccagactccagcctccgtggaggcagctgtgggaggcacagtcaccatcaagtgctcggccagcgcgagcatttacgagtacttatcctggtatcagcagaaaccagggcagcgtcccaagctcctgatctatggtgcgtcggttttaacggatggggtctcatcgcggttcaaaggcagtggatctgggacagagttcactctcaccatcagcgacctggaggctgccgatgctgccacttactactgtcaaagctataatgatggtagtagtagtggcgatgctcatgttttcggcggagggaccgaggtggtggtcaaaggggatccagtggcccccaccgtgctgattttcccaccagccgccgatcaggtcgccaccggcaccgtgacaatcgtgtgcgtggccaacaagtacttccccgacgtgaccgtgacctgggaggtggacggcaccacccagaccaccggcatcgagaacagcaagaccccccagaattctgccgacaacacctacaacctgagcagcaccctgaccctgaccagcacccagtacaacagccacaaagagtacacctgtaaagtcacccagggcaccaccagcgtggtgcagagcttcaaccggggcgactgc

SEQ ID NO: 39 - DNA, 1335 bp, synthetic construct
  source 1..1335: mol_type = other DNA
caggagcagctgaaggagtccgggggaggcctggtccagcctgggggatccctgaaactctcctgcaaagcctctggatttcccttaagtagctatgggtttagctgggtccgccaggctccagggaaggggctggagtggatcggatacctcgaccctgttttcggtagcactttgagcgcccacacggtgaatggccgactcaccatctccagcgacaacgcccagaacacgctgtatctgcaactgaacagtctgacagccgcggacacggccacctatttctgtgcgaggggtatcggttatgtttattatggttatacctatgacttgtggggcccaggcaccctggtcaccgtctcctcaggacagcctaaggccccctccgtgttcccgctagccccttgctgtggcgacaccccttctagcaccgtgacactgggctgcctggtgaagggctacctgcccgagcctgtgaccgtgacctggaacagcggcaccctgaccaacggcgtgagaaccttccctagcgtgcggcagagcagcggcctgtacagcctgagcagcgtggtgtccgtgaccagcagcagccagcccgtgacctgcaatgtggcccaccccgccaccaacaccaaggtggacaagaccgtggcccccagcacctgtagcaagcctacctgcccccctcctgaactgctgggcggacccagcgtgttcatcttcccacccaagcccaaggacaccctgatgatcagccggacccccgaagtgacctgcgtggtggtggacgtgtcccaggacgaccccgaggtgcagttcacctggtacatcaacaacgaacaagtgcggaccgccagacctcctctgcgggagcagcagttcaacagcaccatccgggtggtgtccaccctgcctatcgcccaccaggactggctgcggggcaaagaatttaagtgcaaggtccacaacaaggccctgcctgcccccatcgagaaaaccatcagcaaggccagaggccagcccctggaacctaaggtgtacaccatgggccctcccagagaggaactgagcagcagaagcgtgtccctgacctgcatgatcaacggcttctaccccagcgacatcagcgtggagtgggagaagaacggcaaggccgaggacaactacaagaccacccctgccgtgctggatagcgacggcagctacttcctgtacagcaagctgagcgtgcccacctctgagtggcagcggggcgacgtgttcacctgtagcgtgatgcacgaggccctgcacaaccactacacccagaagtccatcagcagatcccccggcaag

(19) United States
(12) Patent Application Publication
(10) Pub. No.: US 2025/0270309 A1
(43) Pub. Date: Aug. 28, 2025

(54) ANTIBODIES AND METHODS TARGETING INTERLEUKIN-19

(71) Applicant: Eli Lilly and Company, Indianapolis, IN (US)

(72) Inventors: Julian DAVIES, Indianapolis, IN (US); Maya Rachel KARTA, Indianapolis, IN (US); Wei WANG, Indianapolis, IN (US); Shannon Marie HARLAN, Indianapolis, IN (US); Robert Il SIEGEL, Indianapolis, IN (US); Wenyu MING, Indianapolis, IN (US)

(21) Appl. No.: 18/858,293

(22) PCT Filed: Apr. 18, 2023
(86) PCT No.: PCT/US2023/065866

§ 371 Date: Oct. 18, 2024

Related U.S. Application Data
(60) Provisional application No. 63/332,755, filed on Apr. 20, 2022.
(60) Provisional application No. 63/379,450, filed on Oct. 14, 2022.

(51) Int. Cl. C07K 16/24; A61K 39/00

(52) U.S. Cl. CPC C07K 16/244; A61K 2039/505; C07K 2317/20; C07K 2317/76; C07K 2317/92; C07K 2317/94

(57) ABSTRACT

The present invention provides compounds and methods targeting human interleukin-19, including therapeutic antibodies, pharmaceutical compositions and methods of use thereof, useful in the field of immune-mediated diseases including psoriasis, atopic dermatitis, asthma, psoriatic arthritis, rheumatoid arthritis, axial spondyloarthritis, inflammatory bowel disease and colitis.

The present invention is in the field of medicine. More particularly, the present invention relates to antibodies directed against human interleukin-19 (IL-19), pharmaceutical compositions comprising such antibodies, and methods of using such antibodies. The antibodies and methods of the present invention are expected to be useful in the field of autoimmune and chronic inflammatory diseases (collectively referred to herein as, immune-mediated diseases), particularly diseases such as psoriasis (PsO), atopic dermatitis (AD), asthma, psoriatic arthritis (PsA), rheumatoid arthritis (RA), axial spondyloarthritis (AxSpA), inflammatory bowel disease (IBD), colitis, and the like, including treatment thereof.

Interleukin-19 (IL-19) is a cytokine reported to belong to the interleukin-10 cytokine family (which includes IL-10, 20, 22, 24 and 26 as well as some virus-encoded cytokines). IL-19 has been reported to have involvement in the IL-20R complex signaling pathway and to be expressed in resting monocytes, macrophages, B cells, and epithelial cells including keratinocytes. Further, studies have reported IL-19 involvement in immune-mediated diseases (for example, Konrad et al., Scientific Reports 9, Art. No. 5211 (2019) and Steinert et al., J. Immunol 2017; 199:2570-2584 (September 2017)).

Autoimmune diseases are a form of immune-mediated diseases that arise from the body's production of an immune response against its own tissue. Autoimmune diseases are often chronic and can be debilitating and even life-threatening. PsO is a chronic autoimmune disease with systemic manifestations including PsA, cardiovascular disease, metabolic syndrome and affective disorders. AD, along with many other forms of chronic autoimmune diseases such as asthma, PsO, PsA, RA, IBD, colitis and AxSpA, affect the axial and/or peripheral skeleton.

Current FDA approved treatments for immune-mediated diseases include corticosteroids, often used to treat acute inflammation, and bioproducts targeting TNFα, interleukin-12, -17 and -23. Although these treatments have demonstrated efficacy in reducing symptoms for a subset of patients, a percentage of patients remain nonresponsive or experience a loss of response to the currently available treatments. Thus, there remains a need for additional therapies targeting different human targets and pathways for the treatment of immune-mediated diseases.

While IL-19 antibodies are known in the art (WO 2019/143585), to date there are no approved IL-19 antibody therapeutics. Further, nonspecific binding, for example with serum proteins, has proven to be a challenge in the field with regard to IL-19 antibodies. Thus, there remains an unmet need for antibodies, pharmaceutical compositions, and methods which target human IL-19 useful for the treatment of immune-mediated diseases such as AD, asthma, PsO, RA, IBD, colitis, AxSpA, PsA and the like. Such IL-19 antibodies should possess good therapeutic characteristics including being potent neutralizers (i.e., antagonists) of human IL-19, having high binding affinity for human IL-19 and low nonspecific binding including nonspecific binding with serum proteins. Such IL-19 antibodies should also possess therapeutically acceptable pharmacokinetics (Pk) profile and demonstrate low immunogenicity. Such IL-19 antibodies should also be amenable to commercial manufacturing, including high levels of solubility and low levels of aggregation. The present disclosure provides IL-19 antibodies that address these needs for use in the treatment of immune-mediated diseases.

Accordingly, in certain embodiments, the present invention provides antibodies directed against human IL-19. According to some embodiments, the antibodies of the present invention are antagonistic to human IL-19. Embodiments of the present invention provide antibodies which comprise a light chain variable region (LCVR) and a heavy chain variable region (HCVR), wherein the LCVR comprises complementarity determining regions (CDRs) LCDR1, LCDR2 and LCDR3 and the HCVR comprises CDRs HCDR1, HCDR2 and HCDR3, wherein the amino acid sequence of HCDR1 is SEQ ID NO.7, the amino acid sequence of HCDR2 is SEQ ID NO.8, the amino acid sequence of HCDR3 is SEQ ID NO.9 or SEQ ID NO.10, the amino acid sequence of LCDR1 is SEQ ID NO.2, the amino acid sequence of LCDR2 is SEQ ID NO.3, and the amino acid sequence of LCDR3 is SEQ ID NO.4. According to some embodiments, antibodies of the present invention also include antibodies that comprise CDRs having amino acid sequences with at least 95% homology to the amino acid sequences of CDRs herein.

According to some embodiments, the amino acid sequence of HCDR3 is SEQ ID NO.9. According to some such embodiments, the amino acid sequence of the HCVR is SEQ ID NO.11 and the amino acid sequence of the LCVR is SEQ ID NO. 5. According to some embodiments, antibodies of the present invention also include antibodies that comprise LCVRs and HCVRs having amino acid sequences with at least 95% homology to the amino acid sequences of LCVRs and HCVRs herein.

According to some embodiments, the amino acid sequence of HCDR3 is SEQ ID NO.10. According to some such embodiments, the amino acid sequence of the HCVR is SEQ ID NO.13 and the amino acid sequence of the LCVR is SEQ ID NO. 5. According to some embodiments, antibodies of the present invention also include antibodies that comprise LCVRs and HCVRs having amino acid sequences with at least 95% homology to the amino acid sequences of LCVRs and HCVRs herein.

According to some embodiments of the antibodies of the present invention comprise a heavy chain (HC) and a light chain (LC), wherein the amino acid sequence of the HC is SEQ ID NO. 12 or 14 and the amino acid sequence of the LC is SEQ ID NO. 6. In some such embodiments, the amino acid sequence of the HC is SEQ ID NO. 12. In some embodiments, the amino acid sequence of the HC is SEQ ID NO. 14. According to some embodiments, antibodies of the present invention also include antibodies that comprise HCs and LCs having amino acid sequences with at least 95% homology to the amino acid sequences of HCs and LCs herein.

Further embodiments of the present invention include a nucleic acid comprising a sequence encoding SEQ ID NO: 6, 12 or 14. Additional embodiments include a vector comprising a first nucleic acid sequence encoding SEQ ID NO: 12 or 14 and a second nucleic acid sequence encoding SEQ ID NO: 6. Additional embodiments also include a first vector comprising a nucleic acid sequence encoding SEQ ID NO: 12 or 14 and a second vector comprising a nucleic acid sequence encoding SEQ ID NO: 6. Even further embodiments include a cell comprising one or more vectors of the present invention. Further, the present invention provides a process of producing an antibody comprising culturing a cell of the present disclosure under conditions such that the antibody is expressed and recovering the expressed antibody from the culture medium.

Embodiments of the present invention further include an antibody that binds murine IL-19 comprising a heavy chain variable region (HCVR) and a light chain variable region (LCVR), wherein the HCVR comprises complementarity determining regions (CDRs) HCDR1, HCDR2, and HCDR3 and the LCVR comprises CDRs LCDR1, LCDR2, and LCDR3, wherein the amino acid sequence of HCDR1 is SEQ ID NO.21, the amino acid sequence of HCDR2 is SEQ ID NO.22, the amino acid sequence of HCDR3 is SEQ ID NO.23, the amino acid sequence of LCDR1 is SEQ ID NO.16, the amino acid sequence of LCDR2 is SEQ ID NO.17, and the amino acid sequence of LCDR3 is SEQ ID NO.18. According to some embodiments, the amino acid sequence of the HCVR is SEQ ID NO.24 and the amino acid sequence of the LCVR is SEQ ID NO. 19. According to some embodiments, antibodies of the present invention include antibodies comprising a heavy chain (HC) and a light chain (LC), wherein the amino acid sequence of the HC is SEQ ID NO. 25 and the amino acid sequence of the LC is SEQ ID NO. 20. According to some embodiments, antibodies of the present invention also include antibodies that have amino acid sequences with at least 95% homology to the amino acid sequences herein.

Another embodiment of the present invention includes an antibody that binds murine IL-19 comprising a heavy chain variable region (HCVR) and a light chain variable region (LCVR), wherein the HCVR comprises complementarity determining regions (CDRs) HCDR1, HCDR2, and HCDR3 and the LCVR comprises CDRs LCDR1, LCDR2, and LCDR3, wherein the amino acid sequence of HCDR1 is SEQ ID NO.33, the amino acid sequence of HCDR2 is SEQ ID NO.34, the amino acid sequence of HCDR3 is SEQ ID NO.35, the amino acid sequence of LCDR1 is SEQ ID NO.28, the amino acid sequence of LCDR2 is SEQ ID NO.29, and the amino acid sequence of LCDR3 is SEQ ID NO.30. According to some embodiments, the amino acid sequence of the HCVR is SEQ ID NO.36 and the amino acid sequence of the LCVR is SEQ ID NO. 31. According to some embodiments, antibodies of the present invention include antibodies comprising a heavy chain (HC) and a light chain (LC), wherein the amino acid sequence of the HC is SEQ ID NO. 37 and the amino acid sequence of the LC is SEQ ID NO. 32. According to some embodiments, antibodies of the present invention also include antibodies that have amino acid sequences with at least 95% homology to the amino acid sequences herein. Further, according to some embodiments, antibodies of the present invention that bind murine IL-19 do not complete for binding to murine IL-19.

Further embodiments of the present invention include a nucleic acid sequence encoding SEQ ID NO: 26 or 27. Additional embodiments include a vector comprising a first nucleic acid sequence encoding SEQ ID NO:26 and a second nucleic acid sequence encoding SEQ ID NO: 27. Alternatively, the embodiments of the present invention include a nucleic acid sequence encoding SEQ ID NO: 38 or 39. Additional embodiments include a vector comprising a first nucleic acid sequence encoding SEQ ID NO: 38 and a second nucleic acid sequence encoding SEQ ID NO: 39.

Even further embodiments include a cell comprising one or more vectors of the present invention. Further, the present invention provides a process of producing an antibody comprising culturing a cell of the present invention under conditions such that the antibody is expressed and recovering the expressed antibody from the culture medium.

According to some embodiments, a pharmaceutical composition comprising an antibody of the present invention, and one or more pharmaceutically acceptable carriers, diluents or excipients, is provided herein.

Furthermore, methods of treating AD, asthma, PsO, PsA, RA, AxSpA, IBD, colitis or PsA comprising administering to a patient in need thereof an effective amount of an antibody of the present invention, or a pharmaceutical composition of the present disclosure, are provided herein.

Embodiments of the present invention include an antibody of the present invention for use in therapy. According to some embodiments, an antibody of the present invention for use in the treatment of AD, asthma, PsO, PSA, RA, AxSpA, IBD, colitis or PsA is provided herein. Furthermore, an antibody of the present invention for use in the manufacture of a medicament for the treatment of AD, asthma, PsO, PsA, RA, AxSpA, IBD, colitis or PsA is provided herein.

The term “antibody,” as used herein, refers to an immunoglobulin molecule that binds an antigen. Embodiments of an antibody include a monoclonal antibody, polyclonal antibody, human antibody, humanized antibody, chimeric antibody, bispecific or multispecific antibody, or conjugated antibody. The antibodies can be of any class (e.g., IgG, IgE, IgM, IgD, IgA), and any subclass (e.g., IgG1, IgG2, IgG3, IgG4).

An exemplary antibody of the present disclosure is an immunoglobulin G (lgG) type antibody comprised of four polypeptide chains: two heavy chains (HC) and two light chains (LC) that are cross-linked via inter-chain disulfide bonds. The amino-terminal portion of each of the four polypeptide chains includes a variable region of about 100-125 or more amino acids primarily responsible for antigen recognition. The carboxyl-terminal portion of each of the four polypeptide chains contains a constant region primarily responsible for effector function. Each heavy chain is comprised of a heavy chain variable region (VH) and a heavy chain constant region. Each light chain is comprised of a light chain variable region (VL) and a light chain constant region. The IgG isotype may be further divided into subclasses (e.g., IgG1, IgG2, IgG3, and IgG4).

The VH and VL regions can be further subdivided into regions of hyper-variability, termed complementarity determining regions (CDRs), interspersed with regions that are more conserved, termed framework regions (FR). The CDRs are exposed on the surface of the protein and are important regions of the antibody for antigen binding specificity. Each VH and VL is composed of three CDRs and four FRs, arranged from amino-terminus to carboxyl-terminus in the following order: FR1, CDR1, FR2, CDR2, FR3, CDR3, FR4. Herein, the three CDRs of the heavy chain are referred to as “HCDR1, HCDR2, and HCDR3” and the three CDRs of the light chain are referred to as “LCDR1, LCDR2 and LCDR3”. The CDRs contain most of the residues that form specific interactions with the antigen. Assignment of amino acid residues to the CDRs may be done according to the well-known schemes, including those described in Kabat (Kabat et al., “Sequences of Proteins of Immunological Interest,” National Institutes of Health, Bethesda, Md. (1991)), Chothia (Chothia et al., “Canonical structures for the hypervariable regions of immunoglobulins”, Journal of Molecular Biology, 196, 901-917 (1987); Al-Lazikani et al., “Standard conformations for the canonical structures of immunoglobulins”, Journal of Molecular Biology, 273, 927-948 (1997)), North (North et al., “A New Clustering of Antibody CDR Loop Conformations”, Journal of Molecular Biology, 406, 228-256 (2011)), or IMGT (the international ImMunoGeneTics database available on at www.imgt.org; see Lefranc et al., Nucleic Acids Res. 1999; 27:209-212).

Embodiments of the present disclosure also include antibody fragments or antigen-binding fragments that, as used herein, comprise at least a portion of an antibody retaining the ability to specifically interact with an antigen or an epitope of the antigen, such as Fab, Fab′, F(ab′)₂, Fv fragments, scFv antibody fragments, scFab, disulfide-linked Fvs (sdFv), a Fd fragment.

The antibodies of the present invention are monoclonal antibodies. Monoclonal antibodies are antibodies derived from a single copy or clone including, for example, any eukaryotic, prokaryotic or phage clone, and not the method by which it is produced. Monoclonal antibodies can be produced, for example, by hybridoma technologies, recombinant technologies, phage display technologies, synthetic technologies, e.g., CDR-grafting, or combinations of such or other technologies known in the art.

Methods of producing and purifying antibodies are well known in the art and can be found, for example, in Harlow and Lane (1988), Antibodies, A Laboratory Manual, Cold Spring Harbor Laboratory Press, Cold Spring harbor, N.Y., chapters 5-8 and 15, ISBN 0-87969-314-2. For example, mice or rabbits including transgenic mice or rabbits as known in the art, may be immunized with human IL-19, or portions thereof, and the resulting antibodies can be recovered, screened, purified, and the amino acid sequences determined using conventional methods well known in the art.

In particular embodiments of the present invention, the antibody, or the nucleic acid encoding same, is provided in isolated form. As used herein, the term “isolated” refers to a protein, peptide, or nucleic acid which is free or substantially free from other macromolecular species found in a cellular environment.

The antibodies of the present invention may be prepared and purified using known methods. For example, cDNA sequences encoding a HC (for example the amino acid sequence given by SEQ ID NO.12 or 14) and a LC (for example, the amino acid sequence given by SEQ ID NO.6) may be cloned and engineered into a GS (glutamine synthetase) expression vector. The engineered immunoglobulin expression vector may then be stably transfected into CHO cells. As one of skill in the art will appreciate, mammalian expression of antibodies will result in glycosylation, typically at highly conserved N-glycosylation sites in the Fc region. Stable clones may be verified for expression of an antibody specifically binding to human IL-19 (for example, as represented by a recombinantly produced peptide comprising SEQ ID NO. 1). Positive clones may be expanded into serum-free culture medium for antibody production in bioreactors. Media, into which an antibody has been secreted, may be purified by conventional techniques. For example, the medium may be conveniently applied to a Protein A or G Sepharose FF column that has been equilibrated with a compatible buffer, such as phosphate buffered saline. The column is washed to remove nonspecific binding components. The bound antibody is eluted, for example, by pH gradient and antibody fractions are detected, such as by SDS-PAGE, and then pooled. The antibody may be concentrated and/or sterile filtered using common techniques. Soluble aggregate and multimers may be effectively removed by common techniques, including size exclusion, hydrophobic interaction, ion exchange, or hydroxyapatite chromatography. The product may be immediately frozen, for example at −70° C., or may be lyophilized.

The antibodies of the present invention can be used in the treatment of patients. More particularly the antibodies of the present invention are expected to be useful in treating immune-mediated diseases or disorders, which include AD, asthma, PsO, PSA, RA, AxSpA, IBD, colitis and PsA. As used interchangeably herein, “treatment” and/or “treating” and/or “treat” are intended to refer to all processes wherein there may be a slowing, interrupting, arresting, controlling, stopping, or reversing of the progression of the disorders described herein, but does not necessarily indicate a total elimination of all disorder symptoms. Treatment includes administration of an antibody, or pharmaceutical composition thereof, of the present invention for treatment of a disease or condition in a human that would benefit from a reduction in IL-19 activity, and includes: (a) inhibiting further progression of the disease, i.e., arresting its development; or (b) relieving the disease, i.e., causing regression of the disease or disorder, alleviating symptoms or complications thereof, or reduction in “flares” of disease manifestation.

As used interchangeably herein, the term “patient,” “subject,” and “individual,” refers to a human. In certain embodiments, the patient is further characterized with a disease, disorder, or condition (e.g., an immune-mediated disease) that would benefit from a reduction in IL-19 activity. In other embodiments, the patient is further characterized as being at risk of developing an immune-mediated disease, disorder, or condition that would benefit from a reduction in IL-19 activity.

The terms “bind” and “binds” as used herein are intended to mean, unless indicated otherwise, the ability of a protein or molecule to form a chemical bond or attractive interaction with another protein or molecule, which results in proximity of the two proteins or molecules as determined by common methods known in the art.

As referred to herein, the term “epitope” refers to the amino acid residues, of an antigen, that are bound by an antibody. An epitope can be a linear epitope, a conformational epitope, or a hybrid epitope.

The term “epitope” may be used in reference to a structural epitope. A structural epitope, according to some embodiments, may be used to describe the region of an antigen which is covered by an antibody (e.g., an antibody's footprint when bound to the antigen). In some embodiments, a structural epitope may describe the amino acid residues of the antigen that are within a specified proximity (e.g., within a specified number of Angstroms) of an amino acid residue of the antibody.

The term “epitope” may also be used in reference to a functional epitope. A functional epitope, according to some embodiments, may be used to describe amino acid residues of the antigen that interact with amino acid residues of the antibody in a manner contributing to the binding energy between the antigen and the antibody.

An epitope can be determined according to different experimental techniques, also called “epitope mapping techniques.” It is understood that the determination of an epitope may vary based on the different epitope mapping techniques used and may also vary with the different experimental conditions used, e.g., due to the conformational changes or cleavages of the antigen induced by specific experimental conditions. Epitope mapping techniques are known in the art (e.g., Rockberg and Nilvebrant, Epitope Mapping Protocols: Methods in Molecular Biology, Humana Press, 3rd ed. 2018), including but not limited to, X-ray crystallography, nuclear magnetic resonance (NMR) spectroscopy, site-directed mutagenesis, species swap mutagenesis, alanine-scanning mutagenesis, hydrogen-deuterium exchange (HDX) and cross-blocking assays.

An antibody of the present invention can be incorporated into a pharmaceutical composition which can be prepared by methods well known in the art and comprise an antibody of the present invention and one or more pharmaceutically acceptable carrier(s) and/or diluent(s) (e.g., Remington, The Science and Practice of Pharmacy, 22^{nd} Edition, Loyd V., Ed., Pharmaceutical Press, 2012, which provides a compendium of formulation techniques as are generally known to practitioners). Suitable carriers for pharmaceutical compositions include any material which, when combined with an antibody of the present invention, retains the molecule's activity and is non-reactive with the patient's immune system.

A pharmaceutical composition comprising an antibody of the present invention can be administered to a patient at risk for, or exhibiting, diseases or disorders as described herein by parental routes (e.g., subcutaneous, intravenous, intraperitoneal, intramuscular, or transdermal). A pharmaceutical composition of the present invention contains an “effective” or “therapeutically effective” amount, as used interchangeably herein, of an antibody of the present invention. An effective amount refers to an amount necessary (at dosages and for periods of time and for the means of administration) to achieve the desired therapeutic result. An effective amount of an antibody may vary according to factors such as the disease state, age, sex, and weight of the individual, and the ability of the antibody to elicit a desired response in the individual. An effective amount is also one in which any toxic or detrimental effects of the antibody of the present invention are outweighed by the therapeutically beneficial effects.

Percent homology, as used in the present disclosure, in the context of two or more amino acid sequence refers to two or more sequences having a specified percentage of amino acid residues that are the same, when compared and aligned for maximum correspondence, as measured using a sequence comparison algorithm (e.g., BLASTP and BLASTN or other algorithms available to persons of skill) or by visual inspection. Depending on the application, the percent homology can exist over a region of the sequence being compared, e.g., over a functional domain, or, alternatively, exist over the full length of the two sequences to be compared. By way of example, percent homology of a sequence may be compared to a reference sequence. For example, when using a sequence comparison algorithm, test and reference sequences may be input into a computer (and subsequence coordinates may be further designated if desired along with sequence algorithm program parameters). The sequence comparison algorithm then calculates the percent sequence identity or homology for the test sequence(s) relative to the reference sequence(s), based on the designated program parameters. Exemplary sequence alignment and/or homology algorithms are available through, Smith & Waterman, Adv. Appl. Math. 2:482 (1981), Needleman & Wunsch, J. Mol. Biol. 48:443 (1970), Pearson & Lipman, Proc. Nat'l. Acad. Sci. USA 85:2444 (1988), GAP, BESTFIT, FASTA, and TFASTA (in the Wisconsin Genetics Software Package, Genetics Computer Group, 575 Science Dr., Madison, Wis.), or by visual inspection (see generally Ausubel et al., infra). One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

### BRIEF DESCRIPTION OF THE DRAWINGS

The features and advantages of the present invention will become more apparent to those skilled in the art upon consideration of the following detailed description taken in conjunction with the accompanying Figures.

FIG. 1 demonstrates the changes in clinical sum scores over time comparing the vehicle treated mice, anti-inflammatory Ab treated mice and naïve mice in a psoriasis-like mouse model used for gene analysis.

FIGS. 2A and 2B represent the clinical sum scores over time (FIG. 2A) and the area under the curve (FIG. 2B) for a single study evaluating the efficacy of the anti-murine IL-19 Ab4 in a psoriasis-like mouse model.

FIG. 3 demonstrates the increase in ear thickness in the isotype treated mice compared to naïve or anti-inflammatory treated mice in the atopic dermatitis-like mouse model used for gene analysis.

FIG. 4 demonstrates the IL-19 protein levels in the ear during MC903 induced inflammation compared to the naïve group.

FIG. 5 demonstrates the efficacy of the anti-murine IL-19 Ab3 in an atopic dermatitis-like mouse model.

FIGS. 6A and 6B shows the reduction of IL-4 and IL-13 secretion from the ears in the anti-murine IL-19 Ab3 treated mice compared to isotype treated mice.

FIG. 7 compares the change in ear thickness between the isotype control to naïve mice and challenged mice treated with an anti-inflammatory agent in a mouse model of contact dermatitis.

FIG. 8 demonstrates the IL-19 protein levels in the FITC challenged mice when compared to naïve mice.

FIGS. 9 and 10 show a comparison of two separate studies using an anti-murine IL-19 Ab4 in a mouse model of contact dermatitis.

FIG. 11 shows IL-19 expression vs change in ear thickness in an atopic dermatitis-like mouse model of skin inflammation.

FIG. 12 shows IL-19 expression vs clinical sum score area-under-curve in a psoriasis-like mouse model of skin inflammation.

FIG. 13 show IL-19 expression (peak disease vs resolution) in an atopic dermatitis-like mouse model of skin inflammation.

### EXAMPLES

### Expression of Exemplified Anti-IL-19 Antibodies

Exemplified anti-IL-19 antibodies of the present invention are presented in Table 1. cDNA sequences encoding the heavy and light chains of the exemplified anti-IL-19 antibodies of the present disclosure may be cloned and engineered into a GS (glutamine synthetase) expression vector for recombinant expression in a competent cell line, such as CHO cells. The relationship of the various regions of exemplified anti-IL-19 antibodies is as follows (numbering of amino acids applies linear numbering; assignment of amino acids to variable domains is based on the International Immunogenetics Information System® available at www.imgt.org; assignment of amino acids to CDR domains is based on the well-known North numbering convention, with the exception of HCDR2 which C-terminus is based on the well-known Kabat numbering convention).

TABLE 1
SEQ ID NOs of Exemplified Antibody Amino Acid Sequences.

| mAb | HC | HCVR | HCDR1 | HCDR2 | HCDR3 | LC | LCVR | LCDR1 | LCDR2 | LCDR3 |
|---|---|---|---|---|---|---|---|---|---|---|
| Ab1 | 12 | 11 | 7 | 8 | 9 | 6 | 5 | 2 | 3 | 4 |
| Ab2 | 14 | 13 | 7 | 8 | 10 | 6 | 5 | 2 | 3 | 4 |

Exemplified antibodies of the present disclosure are identified as possessing high binding affinity and being chemically and physically stabile including aggregation and solubility consistent with therapeutic parental administration. The exemplified antibodies of the present disclosure are also identified as possessing low immunogenicity (including low nonspecific and/or serum binding) and possessing pharmacokinetic properties consistent with therapeutic parental administration for the treatment of immune-mediated diseases.

### Binding Affinity

A Meso Scale Discovery Solution Equilibrium Titration (MSD-SET) assay, measured with a SECTOR® Imager 6000 (Meso Scale Diagnostics), is used to assess binding of exemplified IL-19 antibodies of the present invention to biotinylated human IL-19 (having the amino acid sequence set for in SEQ ID NO: 1).

Except as noted, all the reagents and materials are from Meso Scale Diagnostics (Rockville, Maryland). Recombinant human IL-19 reagents are biotinylated using the EZ-link sulfo-NHS-Biotin kit (Thermo Cat #A39257). Briefly, 20 pM biotinylated human IL-19 is mixed in a 1:1 ratio with a 3-fold dilution series of exemplified antibody samples of the present invention (mAb 1 and mAb 2) to give a final concentration of: 10 pM biotinylated human IL-19 and a 11-point 3-fold antibody gradient from 1 nM to 0.017 pM. The mix is incubated at 37° C. for 72 hrs. A MSD 96-well plate (Multi-array 96 well plate, Cat #L15XA-3) is coated overnight at 4° C. with 30 μl of 20 nM of exemplified antibodies in phosphate buffered saline (PBS) per well. The plate is then washed three times with 150 μL of wash buffer (0.05% Tween-20 in PBS, VWR Cat #9005-64-5) per well and blocked with 150 μL of blocking buffer (PBS+3% blocker A buffer, Cat. #R93BA-1) per well for 45 min. at 37° C. After three washing cycles, 50 μL per sample of IL-19 and antibody mix is transferred to each well and incubated with shaking (700 rpm) for 150 sec at 37° C. After disposal of samples and three wash cycles, 100 μL of detection antibody (1 μg/mL MSD Sulfo-tag streptavidin antibody, Cat. #R32AD1) is added to each well and incubated with shaking for 3 min at 37° C. After washing three times with wash buffer, 150 μL of 1× Read Buffer T (Cat. #R92TC-1) is added to the well and analyzed on a SECTOR® Imager 6000 (Meso Scale Diagnostics) 5 min after buffer addition. The exemplified antibodies are tested in duplicate within each individual experiment, and three independent experiments were conducted.

The equilibrium K_{D} is determined by fitting a sigmoidal curve to the electrochemiluminescence (ECL) response vs. log (IL-19 concentration) using assay development tool kit graphed with normalized ECL values. The K_{D} is determined as a “curve fit” at 10 pM fixed IL-19 ligands. The average KD values reported are calculated from three independent experiments, and the data variability is expressed in standard deviation (SD) of the 3 independent runs.

TABLE 2
MSD affinity data to recombinant human IL-19.

| Exemplified Antibody | Steady state K_{D} (pM) | SD (pM) | N |
|---|---|---|---|
| Ab_1 | 13.1 | ±1.8 | 3 |
| Ab_2 | 6.8 | ±0.7 | 3 |

K_{D} results are reported as an equilibrium binding at 10 pM fixed biotinylated human IL 19.

Table 2 demonstrates exemplified anti-IL-19 antibodies, mAb1 and mAb2, possess low picomolar binding affinity for human IL-19 in vitro.

### Neutralization of IL-19 In Vitro

Antibodies of the present invention are expected to neutralize IL-19. Neutralization of IL-19 activity by antibodies of the present invention may be assessed by an IL-19/IL-19 receptor (IL20Ra/IL20Rb) dimerization assay format, for example, as described below.

Briefly, U20S cells expressing IL20Ra and IL20Rb (DiscoveRx, cat #93-1027C3) are cultured in Assay Complete Cell Plating Media (DiscoveRx, cat #93-0563R5A). Sub-confluent cells are removed with Assay Complete Cell Detachment Reagent (DiscoveRx, cat #92-0009), spun down, washed, resuspended in Assay Complete Cell Plating Reagent and plated at 2,500 cells per well in 100 μl per well on white-well clear bottom 96-well plate. The cells are cultured for 4 hours followed by treatment with 10 μl per well of 11× mix of antibodies and IL-19. Respective antibody is diluted in DiscoveRx Protein Dilution Buffer (DiscoveRx, cat #92-0023M), starting concentration for antibody 10 μg/ml (1×)) and titration 1:3 for dose response mixed with 22× of either human (SEQ ID NO.1) or cyno (SEQ ID NO. 15) IL-19 (1× equals 100 ng/ml) diluted in DiscoveRx Protein Dilution Buffer. Antibody and ligand mixture is preincubated for 20 minutes; assay medium is used for “no treatment” control and isotype control antibody is used as negative control. Cells are then incubated at 37° C. overnight. PathHunter Flash Detection Kit X (DiscoveRx, cat #93-0247), according to manufacturer instruction, is used for luminescence readout counted at 0.1 to 1 second/well (Perkin Elmer Victor3). Statistical analysis is performed using GraphPad Prism version 9. Percent inhibition from each run, in triplicate, is calculated and means are combined. Data are reported as the IC50 of sigmoidal curve fit (four parameters) and the 95% confidence interval (95% CI, symmetrical). Inhibition of human and cyno IL-19 induced receptor dimerization signal by exemplified anti-IL-19 antibodies is provided in Table 3.

TABLE 3
IL-19 Neutralization in Vitro.

| Exemplified | IC50 for human IL-19 | | IC50 for Cyno IL-19 | |
|---|---|---|---|---|
| Antibody | nM | 95% CI | nM | 95% CI |
| Ab 1 | 3.46 | 2.86 to 4.18 | 2.79 | 2.36 to 3.31 |
| Ab 2 | 3.47 | 3.05 to 3.94 | 2.87 | 2.41 to 3.41 |

### Neutralization of IL-19-Induced Phosphorylated STAT3 Signaling In Vitro

IL-19 neutralization in vitro may be further assessed essentially as described below. A431 dermoid epithelial cells (ATCC #CRL-1555) express IL-19 receptor (IL20Ra/IL20Rb). IL-19 interaction with IL-19 receptor induces phosphorylation of STAT3 in A431 dermoid epithelial cells. Briefly, A431 cells are grown in growth media [HyClone #sh30284.01), 10% HI FBS (Gibco #10082147) and 1×PenStrep (Gibco #15140-122)]. Cells are dissociated in 0.05% trypsin-PBS and plated in serum free media (Opti-MEM (Gibco #31985-070) 0.5% BSA (Gibco #15260-037) and 1×PenStrep at 30,000 cells/100 μl media in 96-well plates (Falcon #353072). Cells are then incubated at 37° C. overnight. Ab 1 or Ab 2, or isotype control, are prepared at a final starting concentration of 20 nM and diluted 2-fold for an 8-point titration. Antibodies are preincubated with either human (SEQ ID NO. 1) or cyno IL-19 (SEQ ID NO. 15) (100 ng/ml) for 20 minutes. Cells are then treated and stimulated with antibody: IL-19 mixture for 15 minutes. Thereafter, media is removed and phosphorylation of STAT3 is determined using the Phospho-STAT3 (Tyr705) Kit (Meso Scale Diagnostics, LLC #K150SVD) following manufacturer's protocol (Note: cells may be frozen after media is removed and prior to phosphorylation determination). Phospho-STAT3 activation is determined by chemiluminescent induction and displayed as relative luminescent units (RLUs) using Meso Scale Diagnostics SECTORImager. Statistical analysis is performed using GraphPad Prism version 9. Percent inhibition of runs done in triplicate are calculated and means are combined. Data are reported as the IC50 of sigmoidal curve fit (four parameters) and the 95% confidence interval (95% CI, symmetrical).

Reduction in human and cyno IL-19 induced phosphor-STAT3 signal by exemplified anti-IL-19 antibodies is shown in Table 4.

TABLE 4
IL-19 Neutralization in Vitro.

| Exemplified | IC50 with human IL-19 | | IC50 with Cyno IL-19 | |
|---|---|---|---|---|
| Antibody | nM | 95% CI | nM | 95% CI |
| Ab 1 | 2.31 | 2.00 to 2.67 | 1.82 | 1.48 to 2.22 |
| Ab 2 | 2.21 | 1.93 to 2.53 | 1.84 | 1.49 to 2.26 |

Tables 3 and 4 demonstrate exemplified anti-IL-19 antibodies, Ab 1 and Ab 2, neutralize both human and cyno IL-19 in vitro.

### In Vivo Target Engagement

Target engagement, in vivo, may be assessed essentially as described below. Briefly, total plasma IL-19 concentration (free IL-19 and IL-19 bound to exemplified IL-19 antibody) is determined substantially as described herein. Biotinylated IL 19 antibody is coated on a streptavidin plate. Exemplified Ab 2 samples are diluted 1:4 in dilution buffer to a concentration of 10 g/mL. A ruthenium labeled anti-IgG4 antibody, which binds Ab 2, is used as a detection antibody.

Baseline plasma IL-19 levels of four cynomolgus monkeys are assessed and measure below the limit of detection (˜20 pg/mL). Thereafter, two cynomolgus monkeys are administered Ab 2 via IV and two cynomolgus monkeys are administered Ab 2 subcutaneously. Thereafter, total plasma IL-19 concentrations are measured. Total plasma IL-19 concentrations demonstrated an increase over time, peaking at 336 h (1651.0 pg/mL and 4467.5 pg/mL for IV; 1382.0 pg/mL and 2127.6 pg/mL for SC). These results demonstrate exemplified IL-19 antibody of the present disclosure engages IL-19 in vivo and target engagement is maintained over time.

### Exemplified Antibody Pharmacokinetics in Vivo

Pharmacokinetic parameters of exemplified IL-19 antibodies of the present disclosure may be assessed essentially as described herein. Briefly, male Sprague Dawley rats are administered a single IV or SC dose of 5 mg/kg Ab1 or Ab2 in PBS (pH 7.4) at volumes of 1 mL/kg. Blood is collected from each animal at 1, 6, 12, 24, 48, 96, 120, 168, 240, 336, 504 and 672 hours post IV dose, or at 3, 6, 12, 24, 48, 96, 120, 168, 240, 336, 504 and 672 hours post SC dose and processed to serum.

Serum concentrations of Ab1 and Ab2 are determined by plate-based total human IgG ELISA. Goat anti-human IgG F(ab′)₂ antibody is coated on the ELISA plate as a capture reagent at 1 mg/mL. After incubation with serum standards, controls or samples, mouse anti-human IgG4 pFc′ horseradish peroxidase (diluted 1:10,000) is used to detect plate-bound Ab1 or Ab2. Pharmacokinetic parameters are calculated using non-compartmental analysis (NCA) for each animal (N=3) and parameters are summarized by the mean and standard deviation (SD), where appropriate. NCA and summary statistic calculations are performed using Phoenix WinNonlin 8.1 or Excel. As shown in Table 5, both Ab1 and Ab2 demonstrate an extended pharmacokinetic profile.

TABLE 5
Plasma Pharmacokinetic Parameters for Exemplified IL-19 Antibodies

| | | C₀ | Cₘₐₓ | Tₘₐₓ | AUC_{0-inf} | CL or CL/F | T_{1/2} | |
|---|---|---|---|---|---|---|---|---|
| mAb | Route | (ng/mL) | (ng/mL) | (hr) | (hr*ng/mL) | (mL/hr/kg) | (hr) | % F |
| Ab1 | IV | 141000 | NA | NA | 23100000 | 0.218 | 275 | NA |
| | | (7690) | | | (2410000) | (0.0228) | (37.2) | |
| Ab1 | SC | NA | 37500 | 96 | 22400000* | 0.223* | 343* | 97 |
| | | | (3450) | (42) | | | | |
| Ab2 | IV | 207000 | NA | NA | 25000000 | 0.203 | 472 | NA |
| | | (17300) | | | (3810000) | (0.0287) | (135) | |
| Ab2 | SC | NA | 43200 | 72 | 21200000 | 0.241 | 265 | 85 |
| | | | (8830) | (42) | (3610000) | (0.0457) | (69.2) | |

*N = 1; Parameters reported as mean or mean (SD), where appropriate; Abbreviations: C₀ = maximum concentration extrapolated to time 0 hour following IV bolus dose, Cₘₐₓ = maximum serum concentration following SC dose, Tₘₐₓ = time of Cₘₐₓ, AUC_{0-inf} = area under the serum concentration time curve from time 0 to infinity, CL = clearance following IV administration, CL/F = apparent CL following SC administration, T_{1/2} = half-life, % F = bioavailability following SC administration calculated as (mean AUC_{0-inf}.SC/mean AUC_{0-inf}.IV)*100%.

### Immunogenicity Analysis

### Dendritic Cell (DC) Internalization

Dendritic cell (DC) internalization assay assesses the internalization of molecules by CD14+ monocytes derived dendritic cells. Briefly, CD14+ monocytes are isolated from periphery blood mononuclear cells (PBMCs) and are cultured and differentiated into DC following standard protocols. PBMCs are isolated using density-gradient centrifugation with Ficoll (#17-1440-02, GE Healthcare) and Sepmate 50 (#15450, STEMCELL Technologies) from LRS-WBC. CD14+ monocytes are isolated using positive selection with a CD14+ microbead kit (#130-050-201, Miltenyi Biotec) following the manufacturer's manual. Cells are then cultured at 1 million/mL with 1000 unit/mL GM-CSF and 600 unit/mL IL-4 for 6 days to drive to immature dendritic cells (MDDC) in RPMI medium with L-glutamine and 25 mM HEPES supplemented with 10% FBS, 1 mM sodium pyruvate, 1× penicillin-streptomycin, 1× non-essential amino acids, and 55 μM 2-mercaptoethanol (complete RPMI medium or medium, purchased from Life Technologies). Medium is changed on day 2 and day 5. On day 6 cells are gently collected with a cell scraper and used for experiment. To obtain mature DCs, cells are treated with 1 μg/mL LPS for 4 hours.

Individual test molecules are normalized to 1 mg/mL with PBS and then further diluted to 8 μg/mL in complete RPMI medium. The detection prob, Fab-TAMRA-QSY7, is diluted to 5.33 μg/mL in complete RPMI medium. The antibody and Fab-TAMRA-QSY7 are mixed with equal volume and incubated for 30 min at 4° C. in dark for complex formation. MDDC are resuspended at 4 million/mL in complete RPMI medium and seeded at 50 μL per well in a 96-well round-bottom plate, to which 50 μL of the antibody/probe complex is added. Cells are incubated for 24 h at 37° C. in a CO2 incubator. Cells are washed with 2% FBS PBS and resuspended in 100 μL 2% FBS PBS with Cytox Green live/dead dye. Data are collected on a BD LSR Fortessa X-20 and analyzed in FlowJo. Live single cells are gated and percent of TAMRA fluorescence positive cells is recorded as the readout. To allow the comparison of molecules with data generated from different donors, a normalized internalization index is used. The internalization signal is normalized to IgG1 isotype (normalized internalization index=0) and an internal positive control PC (normalized internalization index=100) using the formula:

$$100 \times \frac{{X}_{TAMRA} - IgG 1 {isotype}_{TAMRA}}{{PC}_{TΛMRΛ} - IgG 1 {isotype}_{TΛMRΛ}}$$

where X_{TAMRA}, IgG1 isotype_{TAMRA}, and PC_{TAMRA} are the percent of TAMRA-positive population for the test molecule X, IgG1 isotype, and PC respectively.

TABLE 6
DC Internalization Assessment for Exemplified IL-19 Antibodies

| Exemplified Antibody | Internalization Index |
|---|---|
| Ab1 | 9.5 |
| Ab2 | 10.3 |

For normalized internalization index, 0-15 is considered low, >15-30 is considered low to moderate, >30-60 is considered moderate, and >60 is considered high risk of immunogenicity.

Table 6 demonstrates exemplified anti-IL-19 antibodies, Ab1 and Ab2, possess low risk of DC internalization.

### MHC-Associated Peptide Proteomics

MHC-associated peptide proteomics (MAPPs) profiles the human leukocyte antigen class II (HLA-II) presented peptides on human dendritic cells previously treated with test molecule to assess immunogenicity. Briefly, primary human dendritic cells from a panel of 10 normal human donors are prepared from buffy coats by isolation of CD-14 positive cells and differentiated into immature dendritic cells by incubation with 20 ng/ml IL-4 and 40 ng/ml GM-CSF in complete RPMI media containing 5% Serum Replacement (Thermo Fisher Scientific, cat #A2596101) for 3 days at 37° C. and 5% CO2 as described (Knierman et al., 2020). Three micromolar of test antibody is added to approximately 5×10⁶ cells on day 4 and fresh media containing 5 μg/ml of LPS to transform the cells into mature dendritic cells is exchanged after 5-hour incubation. Matured cells are lysed in 1 mL of RIPA buffer with protease inhibitors and DNAse the following day. Lysates are stored at −80° C. until sample analysis.

An automated liquid handling system is used to isolate HLA-II molecules from thawed lysate using biotinylated anti-pan HLA class II antibody (clone Tu39). Bound receptor-peptide complex is eluted with 5% acetic acid, 0.1% TFA. Eluted HLA-II peptides are passed over a prewashed 10 k MWCO filter to remove high molecular weight proteins. Isolated HLA-II peptides are analyzed by nano LC/MS using a Thermo easy 1200 nLC-HPLC system with a Thermo LUMOS mass spectrometer. Separation uses a 75 μm×7 cm YMC-ODS C18 column for 65-minute gradient with a 250 nL/min flow rate and 0.1% formic acid in water as A solvent and 80% acetonitrile with 0.1% formic acid as B solvent. Mass spectrometry is run in full scan mode with 240,000 resolution followed by a 3 second data dependent MS/MS cycle comprised of ion trap rapid scans with HCD and EThcD fragmentation.

Peptide identifications are generated by an internal proteomics pipeline (Higgs et al. 2008) using multiple search algorithms with no enzyme search parameter against a bovine/human database containing the test molecule sequence. Peptides identified from the test molecules are aligned against the parent sequence. A summary is created for all test molecules that annotates the percent of donors that display peptides with non-germline residues and the number of different regions of the test molecule that display peptides with non-germline residues. Increases in the extent of display of non-germline peptides is associated with increased risk for immunogenicity.

TABLE 7
MAPPs Results for Exemplified IL-19 Antibodies

| | Number regions with non- | Percentage of donors |
|---|---|---|
| Exemplified | germline residues across | displaying non-germline |
| Antibody | all donors | regions |
| Ab1 | 1 | 40% |
| Ab2 | 1 | 20% |

Table 7 demonstrates exemplified anti-IL-19 antibodies, Ab1 and Ab2, possess low risk MAPPs profiles.

### T Cell Proliferation Assay

T cell proliferation assay assesses the ability of an exemplified antibody to activate CD4+ T cells by inducing cellular proliferation. Briefly, cryopreserved PBMC's are used from 10 healthy donors and the CD8+ T cells are depleted from the PBMC's and labeled with 1 μM Carboxyfluorescein Diacetate Succinimidyl Ester (CFSE). PBMCs are seeded at 4×10⁶ cells/ml/well in AIM-V media (Life Technologies, cat #12055-083) containing 5% CTS™ Immune Cell SR (Gibco, cat #A2596101) and tested in triplicate in 2.0 mL containing the different exemplified antibody, DMSO control, media control, keyhole limpet haemocyanin (KLH; positive control). Cells are cultured and incubated for 7 days at 37° C. with 5% CO2. On day 7, samples are stained with the following cell surface markers: anti-CD3, anti-CD4, anti-CD14, anti-CD19, and DAPI for viability detection by flow cytometry using a BD LSRFortessa™, equipped with a High Throughput Sampler (HTS). Data is analyzed using FlowJo® Software (FlowJo, LLC, TreeStar) and a Cellular Division Index (CDI) is calculated. The CDI for each exemplified antibody is calculated by dividing the percent of proliferating CFSE^{dim}CD4+ T cells from antibody-stimulated wells by the percent of proliferating CFSE^{dim}CD4+ T cells in the unstimulated wells. A CDI of ≥2.5 is considered to represent a positive response. A percent donor frequency across all donors is evaluated.

TABLE 8
T cell Proliferation Results for Exemplified IL-19 Antibodies

| | | Median | Median | | | Number of |
|---|---|---|---|---|---|---|
| | % | CDI | CDI | | | positive |
| Exemplified | Positive | (Positive | (All | Range | | donors |
| Antibody | Donors | Donors) | donors) | High | Low | (CDI > 2.5) |
| Ab1 | 20 | 9 | 1.1 | 15.2 | 0.4 | 2/10 |
| Ab2 | 10 | 2.8 | 1.1 | 2.8 | 0.5 | 1/10 |

Proliferation of <30% donors is considered low; 30-40% moderate; >40% high risk for immunogenicity.

Table 8 demonstrates exemplified anti-IL-19 antibodies, Ab1 and Ab2, possess low risk T cell proliferation immunogenicity profiles.

### Pre-Existing Reactive Assay

Pre-existing reactive assay assesses the presence of reactivity derived from pre-existing anti-drug antibodies (PEA), and potentially other cross-reactive proteins, in treatment naïve normal human serum. Briefly, diluted serum from a panel of at least 50 treatment naïve donors is captured overnight on a plate coated with biotinylated exemplified antibody. On the following day, captured reactive proteins are acid eluted and then neutralized in the presence of biotinylated and ruthenylated exemplified antibody. If anti-drug antibodies are present, a complex will form of exemplified antibody. The complex is captured by a streptavidin-coated Mesoscale plate, and the resulting signal is referred to as Tier 1 signal (expressed as electrochemiluminescence). Tier 1 signal is confirmed in Tier 2 by adding excess unlabeled exemplified antibody in the detection step, which results in the suppression of the Tier 1 signal. The presence of pre-existing anti-drug antibodies is expressed as the 90th percentile of Tier 2 inhibition.

TABLE 9
Pre-Existing Reactivity Results for Exemplified IL-19 Antibodies

| Exemplified Antibody | 90^{th} Percentile Tier 2 Inhibition |
|---|---|
| Ab1 | 20.3 |
| Ab2 | 26.1 |

Results <30% are low, 30%-55% moderate, and >55% high risk for immunogenicity.

Table 9 demonstrates exemplified anti-IL-19 antibodies, Ab1 and Ab2, possess low risk from PEA reactivity.

### Physical-Chemical Properties of Exemplified Antibodies

The exemplified antibodies, Ab1 and Ab2, demonstrate solubility, low aggregation, chemical stability and physical stability characteristics essential for parental therapeutic administration.

### Solubility:

Sufficiently high solubility is desired to enable convenient dosing. For example, a 1 mg/kg dose administered by a 1.0 mL injection into a 100 kg patient will require solubility of 100 mg/ml. In addition, maintaining the antibody in monomeric state without high molecular weight (HMW) aggregation at high concentration is also desirable. Solubility of the exemplified antibodies is analyzed by concentrating 15 mg of an exemplified antibody with a 10 K molecular weight cut-off filter (Amicon U.C. filters, Millipore, catalog #UFC903024) to a volume of less than 100 μl. The final concentration of the sample was measured by UV absorbance at A280 using a Nanodrop 2000 (Thermo Scientific). Following procedures substantially as described above, the exemplified antibodies display a solubility of greater than 200 mg/ml (at pH 7.4 in PBS buffer). In addition, only low levels of HMW (from ˜3.8 to ˜4.35%) are present at high concentration and no phase separation is observed.

### Chemical and Physical Stability:

Chemical stability facilitates the development of drug formulations with sufficient shelf-life. Chemical stability of the exemplified antibodies is assessed by formulating the exemplified antibodies to a concentration of 100 mg/ml in a buffered solution, pH 6. Formulated samples are incubated for four weeks at 4° C., and 35° C. in an accelerated degradation study. Changes in the antibody, reflecting chemical changes, are assessed using CE-SDS and aSEC according to standard procedures. Following procedures substantially as described above, the exemplified antibodies demonstrate chemical stability results presented in Table 10.

TABLE 10
Summary of change in % main peak over four weeks,
relative to samples incubated at 4° C., measured
by CE-SDS and % HMW aggregates measured by aSEC.

| | Change in % of | Change in |
|---|---|---|
| | main peak after 4 | % HMW |
| | weeks (relative to | aggregates |
| | 4° C.) | (relative to 4° C.) |
| Sample | 35° C. | 35° C. |
| Ab 1 | −0.6 | 0.9 |
| Ab 2 | −1.9 | 0.2 |

Results provided in Table 10 demonstrate that after 4 weeks storage at 35° C., the exemplified antibodies have a percentage of main peak decrease of around 0.6 to 1.9%. In addition, mass spectrometry analysis demonstrates only minimal degradation observed after 4 weeks storage at 35° C. (˜0.2% CDRs PTM changes in all CDR sequences), indicating that the exemplified antibodies have sufficient chemical stability to facilitate development of solution formulations with adequate shelf life.

### IL-19 Expression and Activity in Psoriasis-Like Mouse Model

Female BALB/c mice (Envigo, Inc., Indianapolis, IN) at 8 weeks of age were maintained on an ad lib access to food and water. Thirty mg of 3.75% imiquimod (IMQ) cream (Zyclara) (Bausch Health Companies Inc., Laval, Quebec, Canada) was applied daily to the shaved back (2×2 cm region) of mice. Mice were randomized based on body weight into treatment groups with 6 mice in each group. Mice were given an isotype control/vehicle, an anti-inflammatory positive control antibody, or an anti-murine IL-19 Ab (“mAb4”, comprising HCVR of SEQ ID NO. 36 and LCVR of SEQ ID NO. 31) subcutaneously one day before IMQ application. Clinical scores were given 0-4 based on none, slight, moderate, marked, or severe on erythema, thickness and scaling changes with 12 being the maximal sum score. All mice were sacrificed on day 8.

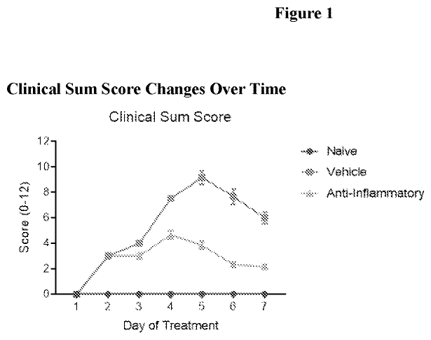

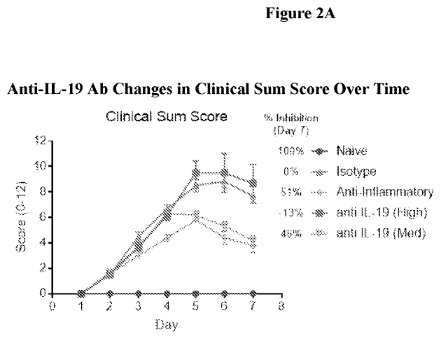

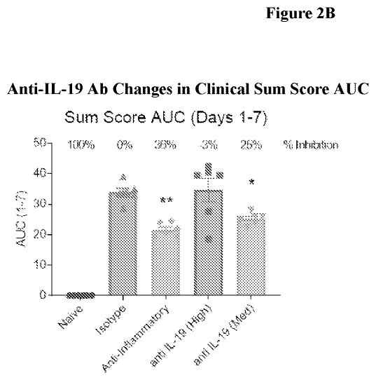

FIG. 1 demonstrates the changes in clinical sum scores over time comparing the vehicle treated mice, anti-inflammatory Ab treated mice and naïve mice. This study was used for further gene analysis to evaluate IL-19 mRNA levels in this model. FIGS. 2A and 2B represent the clinical sum scores over time (FIG. 2A) and the area under the curve (FIG. 2B) for a single study evaluating the efficacy of mAb4 in this model. mAb4 at the dose of 10 mg/kg improved sum clinical score by 25% and reduced last day clinical score by 46% when compared to the isotype control. However, the mice in the high treatment group of 20 mg/kg had no improvement in sum clinical scores when compared to isotype control.

This study demonstrates that anti-IL-19 antibodies have potential to treat psoriasis-like skin diseases. In this regard, the results demonstrate that the antibodies of the present invention reduce inflammation in murine inflammatory models.

### IL-19 Expression and Activity in Atopic Dermatitis-Like Mouse Model

Balb/cJ aged 6-8 weeks were anesthetized by inhalation with isoflurane (5%) and the thickness of both ears were measured using digital calipers and recorded for baseline ear measurement. On day 1, 10 μL of MC903 (Tocris Bioscience) was applied to both the dorsal and ventral sides of each ear with a pipette (total of 40 μL). Following the application of MC903, mice remained under anesthesia until the treatment area was dry. Ear measurements are recorded two or three times per week. Left and right ear thickness (inflammation) was averaged for each mouse daily and the change in thickness was calculated and recorded between measurements. The standard protocol has a total of four challenges performed on days 1, 4, 6, & 8. Mice were dosed on days 0 and 7 with 10 mg/kg of either an isotype control Ab, an anti-inflammatory agent as a positive control, or an anti-murine IL-19 Ab (“mAb 3”, comprising HCVR of SEQ ID NO. 24 and LCVRs of SEQ ID NO. 19). Ears were prepared for culture by manual separation of dermal layers with forceps and placing into RPMI culture media. IL-19 levels in the ear culture are measured by an ELISA developed in-house, using mAb3 and mAb4 that do not compete for binding with each other. The IL-4 and IL-13 levels from the ear cultures were assessed using a custom U-Plex Biomarker MSD Group 1 according to the manufacturer's protocol (Meso Scale Discovery). Data analysis and statistical significance is done using GraphPad Prism Version 9.

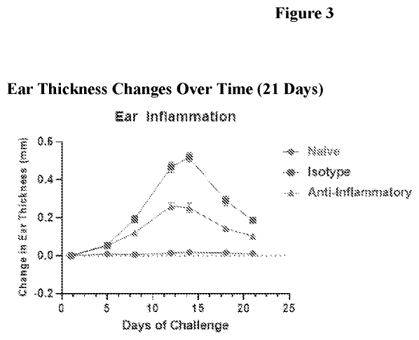

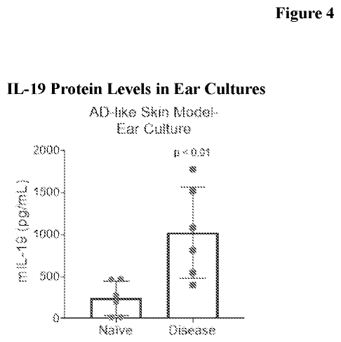

FIG. 3 results compare the isotype control to normal non-challenged and no treatment mice up to the peak of inflammation at day 14. The isotype controls have no effect on the inflammatory response measured by ear thickness with precision electronic calipers. Non-treated and no challenge controls show no inflammatory response. Treatment with a known anti-inflammatory agent dosed weekly for two weeks (day 0 & 7) at 10 mg/kg significantly reduces the inflammatory effect of MC903 challenges and is sustained out to day 21 (FIG. 3). These studies were used for further gene analysis to evaluate IL-19 mRNA levels in this model. FIG. 4 demonstrates that the IL-19 protein levels in the ear are significantly elevated during MC903 induced inflammation compared to the naïve group, confirming the elevation of IL-19 in this model of skin inflammation.

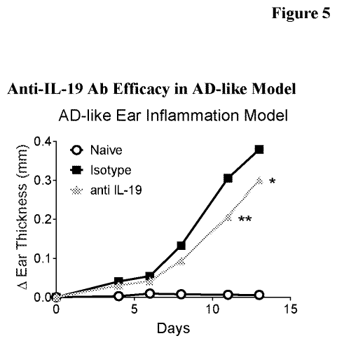

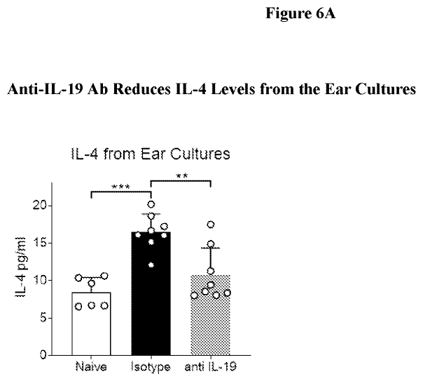

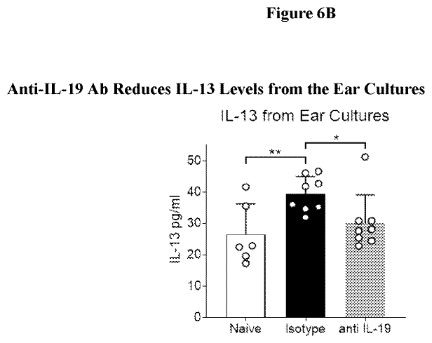

FIG. 5 demonstrates the efficacy of “mAb 3”, comprising HCVR of SEQ ID NO. 24 and LCVRs of SEQ ID NO. 19 in this model. mAb3 significantly reduced ear thickness on days 11 and 13 compared to the isotype control treated mice. In addition, isotype treated mice have elevated IL-4 and IL-13 secretion from the ears compared to the naïve untreated mice (FIGS. 6A and 6B). mAb3 reduced both IL-4 and IL-13 secretion in the ear cultures compared to the isotype group. This demonstrates that mAb3 not only reduces ear inflammation but also reduces Th2 cytokine production in this AD-like mouse model of inflammation. The results demonstrate that the antibodies of the present invention reduce inflammation in murine inflammatory models.

### IL-19 Expression and Activity in a Contact Dermatitis Mouse Model

C57BL/6J aged 6-8 weeks were anesthetized by inhalation with isoflurane (5%) and the thickness of both ears were measured using digital calipers and recorded for baseline ear measurement. Twenty-four hours following the challenge, or day 7, ears were measured again. On Day 0, while under inhaled 5% isoflurane anesthesia, all mice were shaved on the ventral side. 100 μl of Fluorescein isothiocyanate isomer I (FITC; Sigma) in DBP: acetone was applied to the shaved area. The mouse remained under anesthesia until the solution had dried. On Day 1, the dosing and application of FITC in DBP: acetone procedure was repeated. Mice were treated with either an isotype control Ab or an anti-murine antibody, mAb4 (comprising HCVR of SEQ ID NO. 36 and LCVR of SEQ ID NO. 31) on Day 5. On Day 6, after the baseline measurements were taken, mice are challenged with 10 μL of FITC in DBP: acetone solution and applied to both sides of both ears for a total of 40 μL per mouse. Ears were prepared for culture by manual separation of dermal layers with forceps and placing into RPMI culture media. IL-19 levels in the ear culture were measured by an ELISA developed in-house, using mAb3 and another anti-murine IL-19 Ab that do not compete for binding with each other. Data analysis and statistical significance is done using GraphPad Prism Version 9

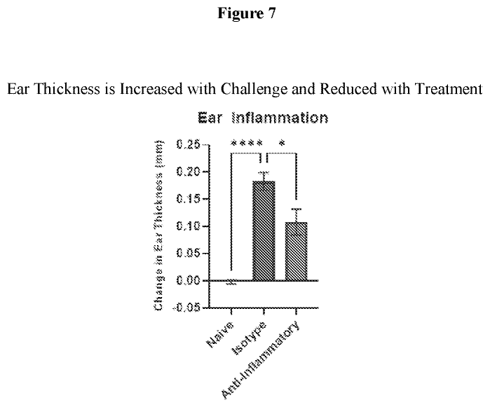

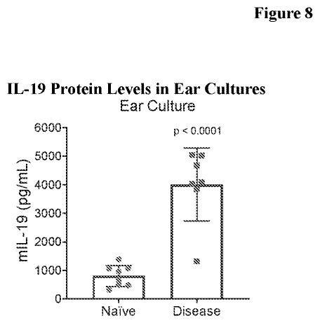

FIG. 7 results compare the isotype control to naïve mice and challenged mice treated with an anti-inflammatory agent up to the peak of inflammation at day 7. The isotype controls have no effect on the inflammatory response measured by ear thickness with precision electronic calipers. Naive no challenge controls show no inflammatory response. Treatment with an anti-inflammatory agent dosed at 10 mg/kg for 24 hours prior to the challenge significantly reduces the inflammatory effect of FITC challenge. In addition, FIG. 8 demonstrates that IL-19 protein levels are elevated in the FITC challenged mice when compared to naïve mice, confirming the elevation of IL-19 in this model of skin inflammation.

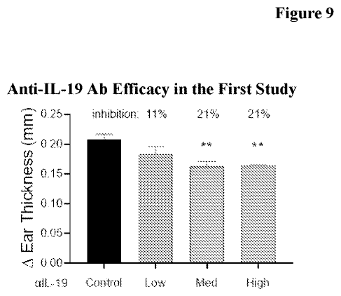

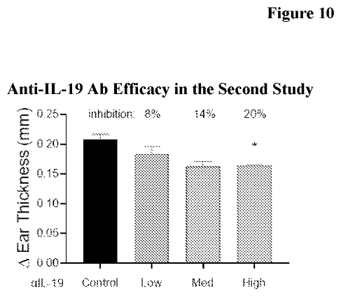

FIGS. 9 and 10 are the results comparing two separate studies using mAb4 in this model. In FIG. 9, both the high (20 mg/kg) and medium (10 mg/kg) doses of mAb4 significantly reduced ear thickness compared to the isotype treated group (21% inhibition), while the low dose (3 mg/kg) did not significantly reduce ear thickness (11% inhibition). In a repeated study (FIG. 10), mAb4 significantly reduced ear thickness at the high dose (20% inhibition), but not at the medium (10 mg/kg) and low doses (3 mg/kg) compared to the isotype control group. Taken together, this demonstrates that at the high dose (30 mg/kg) mAb4 reduces the inflammation induced in this contact dermatitis model. The results demonstrate that the antibodies of the present invention reduce inflammation in murine inflammatory models.

### IL-19 Gene Expression in In Vivo Models of Skin Inflammation

To assess changes in IL 19 gene expression in these mouse models of skin inflammation qPCR and NanoString analysis were used to evaluate the tissue RNA levels. Isolation of RNA from selected tissues was performed using the Qiagen RNeasy (Cat #74181) and performed according to the manufacturer's suggested protocol for animal tissues using the spin protocol.

Gene expression levels were determined by two-step TaqMan RT-PCR using a custom 96-marker gene expression panel and the Viia-7 platform (Applied Biosystems). RNA previously purified from mouse tissue was first reverse-transcribed into cDNA using the Qiagen QuantiTect Reverse Transcription kit following manufacturer recommended methods. Resulting cDNA was diluted 1:10 for downstream applications. RT-PCR is performed according to the manufacturer recommended methods for AppliedBiosystems TaqMan Custom Plates. Gene expression levels are determined by NanoString using the “Mouse Cancer Immune Profiling Panel” following manufacturer recommended protocols. Previously purified RNA was diluted to 20 ng/ul in RNase free water, and a total of 100 ng (in 5 μl) was used to prepare the NanoString cartridges. Raw mRNA counts were normalized using the nSolver Advanced Analysis software (Version 2.0.115) following manufacturer recommended data processing methods. All mRNA counts were initially scaled to intra-sample binding density controls, then experimental genes were further normalized to a housekeeping gene index following dynamic housekeeping gene selection for the entire data set, minimizing housekeeper variance. Normalized gene counts were then compared.

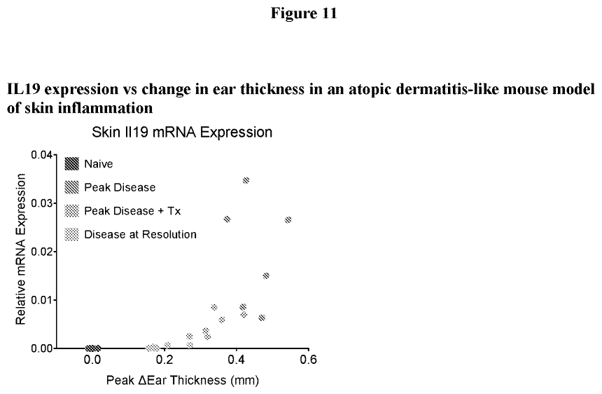

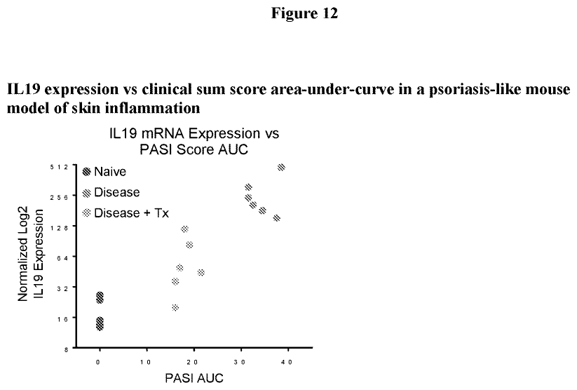

IL19 gene expression was assessed by qRT-PCR or NanoString and found to be elevated in mouse models of skin inflammation representative of atopic dermatitis and psoriasis (FIGS. 1 and 3). Increases in IL19 gene expression correlated strongly with measurements of disease pathology in tissue such as increased ear thickness in the atopic dermatitis-like model (FIG. 11) and psoriasis clinical sum scores (FIG. 12).

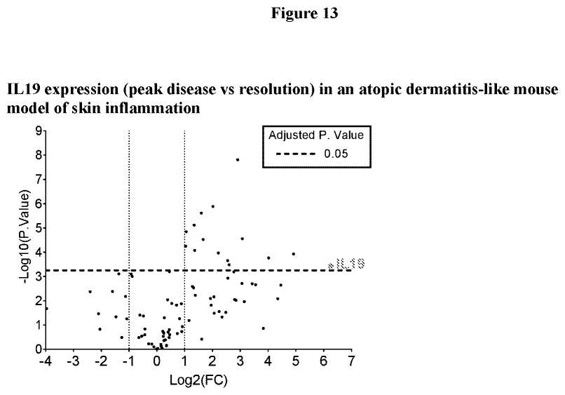

Additionally, treatment significantly reduced IL19 gene expression commensurate with the changes in tissue pathology observed in the same tissue (FIGS. 1 and 3). In the atopic dermatitis-like model, IL19 mRNA was the single most elevated transcript observed by differential expression analysis of the genes evaluated in this study (FIG. 13). Taken together these findings indicate that IL19 gene expression is strongly associated with tissue inflammation in skin diseases.

### Preparation of Anti-Murine IL-19 Antibodies (mAb 3 and mAb 4)

Rabbits are immunized with mouse IL-19. Splenocytes are obtained and a library derived from the splenocytes is constructed by amplifying the variable heavy (VH) and variable light (VL) genes and combining into single-chain Fab for expression using yeast cell surface display. mAb 3 and mAb 4 were obtained after panning the library with mouse IL-19. The sequences of the clones were determined and used to construct expression plasmids for recombinant IgG expression.

Both mAb3 and mAb4 are expressed as recombinant rabbit IgG after co-transfection into CHO cells and purification using MabSelect (Protein A) resin.

1. An antibody that binds human IL-19 comprising a heavy chain variable region (HCVR) and a light chain variable region (LCVR), wherein the HCVR comprises complementarity determining regions (CDRs) HCDR1, HCDR2, and HCDR3 and the LCVR comprises CDRs LCDR1, LCDR2, and LCDR3, wherein the amino acid sequence of HCDR1 is SEQ ID NO. 7, the amino acid sequence of HCDR2 is SEQ ID NO. 8, the amino acid sequence of HCDR3 is SEQ ID NO. 9 or SEQ ID NO. IO, the amino acid sequence of LCDR1 is SEQ ID NO. 2, the amino acid sequence of LCDR2 is SEQ ID NO. 3, and the amino acid sequence of LCDR3 is SEQ ID NO. 4

2. The antibody of claim 1, wherein HCDR3 is SEQ ID NO. 9.

3. The antibody of claim 2, wherein the amino acid sequence of the HCVR is SEQ ID NO. 11 and the amino acid sequence of the LCVR is SEQ ID NO. 5.

4. The antibody of claim 1, wherein HCDR3 is SEQ ID NO. 10.

5. The antibody of claim 4, wherein the amino acid sequence of the HCVR is SEQ ID NO. 13 and the amino acid sequence of the LCVR is SEQ ID NO. 5.

6. An antibody comprising a heavy chain (HC) and a light chain (LC), wherein the amino acid sequence of the HC is SEQ ID NO. 12 or 14 and the amino acid sequence of the LC is SEQ ID NO. 6.

7. The antibody of claim 6, wherein the amino acid sequence of the HC is SEQ ID NO. 12.

8. The antibody of claim 6, wherein the amino acid sequence of the HC is SEQ ID NO. 14.

9. A pharmaceutical composition comprising the antibody of claim 1 and one or more pharmaceutically acceptable carriers, diluents or excipients.

10. A method of treating AD, asthma, PsO, PsA, RA, AxSpA, IBD, colitis or PsA comprising administering to a patient in need thereof an effective amount of the pharmaceutical composition of claim 9.

11. The antibody of claim 1 for use in therapy.

12. The antibody of claim 1 for use in the treatment of AD, asthma, PsO, PsA, RA, AxSpA, IBD, colitis or PsA.

13. The antibody of claim 1 for use in the manufacturer of a medicament for the treatment of AD, asthma, PsO, PsA, RA, AxSpA, IBD, colitis or PsA.

14. A nucleic acid comprising a sequence encoding SEQ ID NO: 6, 12 or 14.

15. A vector comprising a first nucleic acid sequence encoding SEQ ID NO 12 or 14 and a second nucleic acid sequence encoding SEQ ID NO: 6.

16. A cell comprising the vector of claim 15.

17. A composition comprising a first vector comprising a nucleic acid sequence encoding SEQ ID NO: 12 or 14 and a second vector comprising a nucleic acid sequence encoding SEQ ID NO: 6.

18. A cell comprising the first vector and second vector of claim 17.

19. A process of producing an antibody comprising culturing the cell of claim 16 under conditions such that the antibody is expressed and recovering the expressed antibody from the culture medium.

20. An antibody produced by culturing the cell of claim 16 under conditions such that the antibody is expressed and recovering the expressed antibody from the culture medium.

21. An antibody that binds human IL-19 comprising a heavy chain variable region (HCVR) and a light chain variable region (LCVR), wherein the HCVR comprises complementarity determining regions (CDRs) HCDR1, HCDR2, and HCDR3 and the LCVR comprises CDRs LCDR1, LCDR2, and LCDR3, wherein the amino acid sequence of HCDR1 is SEQ ID NO. 21, the amino acid sequence of HCDR2 is SEQ ID NO. 22, the amino acid sequence of HCDR3 is SEQ ID NO. 23, the amino acid sequence of LCDR1 is SEQ ID NO. 16, the amino acid sequence of LCDR2 is SEQ ID NO. 17, and the amino acid sequence of LCDR3 is SEQ ID NO. 18.

22. The antibody of claim 21, wherein the amino acid sequence of the HCVR is SEQ ID NO. 24 and the amino acid sequence of the LCVR is SEQ ID NO. 19.

23. An antibody comprising a heavy chain (HC) and a light chain (LC), wherein the amino acid sequence of the HC is SEQ ID NO. 25 and the amino acid sequence of the LC is SEQ ID NO. 20.

24. An antibody that binds human IL-19 comprising a heavy chain variable region (HCVR) and a light chain variable region (LCVR), wherein the HCVR comprises complementarity determining regions (CDRs) HCDR1, HCDR2, and HCDR3 and the LCVR comprises CDRs LCDR1, LCDR2, and LCDR3, wherein the amino acid sequence of HCDR1 is SEQ ID NO. 33, the amino acid sequence of HCDR2 is SEQ ID NO. 34, the amino acid sequence of HCDR3 is SEQ ID NO. 35, the amino acid sequence of LCDR1 is SEQ ID NO. 28, the amino acid sequence of LCDR2 is SEQ ID NO. 29, and the amino acid sequence of LCDR3 is SEQ ID NO. 30.

25. The antibody of claim 24, wherein the amino acid sequence of the HCVR is SEQ ID NO. 36 and the amino acid sequence of the LCVR is SEQ ID NO. 31.

26. An antibody comprising a heavy chain (HC) and a light chain (LC), wherein the amino acid sequence of the HC is SEQ ID NO. 37 and the amino acid sequence of the LC is SEQ ID NO. 32.

27. The antibody of claim 21 wherein the antibody is an anti-murine IL-19 antibody.
